(19) United States
(12) Patent Application Publication
(10) Pub. No.: US 2003/0123048 A1
(43) Pub. Date: Jul. 3, 2003

(54) On Line crude oil quality monitoring method and apparatus

(76) Inventor: Yaosheng Chen, Blacksburg, VA (US)

Correspondence Address: Yaosheng Chen, 200 Country Club Drive Bldg D, Blacksburg, VA, 24060, US

(21) Appl. No.: 10/032,791

(22) Filed: Dec. 28, 2001

(51) Int. Cl. G01N 33/28

(52) U.S. Cl. 356/70

(57) ABSTRACT

A method and an apparatus for monitoring the quality of crude oil, including oil, gas, and water holdup, are disclosed on-line. The apparatus comprises a series of fiber optical sensors that are installed into the transferring pipe of the crude oil, which exits at any given moment from underground. The method provides analytical data on-line concerning the status of the crude oil at any point in time. Great reliability and a quick response can be expected without sending samples to a laboratory. Therefore, the invented method allows the user to adjust the technology of the oil extraction according to the status of the crude oil.

### FIELD OF INVENTION

The present invention relates to the optics of fiber sensors, optoelectric technology, and on-line monitoring technology. The method can be utilized in a single station of oil extraction or a united station that includes numerous branch stations. The station(s) will serve as the database to decipher the technology of the oil station. Oil quality control in an oil refinery, a central oil storage tank, or a power plant employing heavy oil as fuels can be obtained by the present invention as well. The method invented will also be suitable for water ratio analysis including all types of fluids, such as milk.

### BACKGROUND

Once oil extractions are relayed from oil wells, data will be conveyed in an analytical laboratory is the present method at hand. The present method offers inaccurate data, due to hydrocarbons polluting the oil samples; which evaporate when exposed to air in the atmosphere. The ray method is currently an available on-line method for quality analysis of crude oil. However, the method confronts many issues, such as high cost, safety, and heavy volume holding. In comparison with above existing methods, the present invention allows innumerable advantages: minimum cost, minute structure, greater efficiency, accuracy and installation with ease. The apparatus invented is installed in each oil well and functions with only one end-control. The present invention proves to be a valuable tool in the intellectual setup process, and allows minimum management in the modem technology world of oil extraction.

The single fiber optical sensor, which is employed to analyze oil, water and gas, is commercially available, however, the present invention is an on-line analytical system composed from a series of such sensors. The invented apparatus is applied at an oil extraction station, and is installed into the exit lines of each oil well before the oils are combined. This method proves to be an efficient way to monitor the quality of the crude oil at any point in time to ensure that the petroleum industry reaps maximum economical benefits.

### BRIEF DESCRIPTION OF THE DRAWINGS

FIG. 1 shows a schematic block diagram of the on-line oil quality monitoring apparatus. In the first unit, the signal of oil/water/gas ratio, obtained from the fiber optical sensor, is subjected to optoelectric exchange. After pre-amplification has occurred, transmission to a simulated signal takes place as soon as the sample is extracted. The output of the second unit is pattern discrimination, which is transmitted to the third unit. The third unit deals with statistics, storage, refurbishing, and the feedback of signals, which is monitored on-line. A cable connects the units.

The structure of the fiber optical sensor is shown in FIG. 2.

### REFERENCE NUMERALS IN DRAWINGS

1. Gem probe

2. Probe stand

3. Sensor stand

4. Washer

5. Stainless pipe

6. Lead washer

7. Nut

8. Optical fiber

9. Stainless steel sleeve

10. Red copper washer

11. Flange plate

12. Silicon rubber washer

13. Box of circuit plate

14. Circuit plate

15. Optoelectric transfer stand

16. Plug socket

17. Cable

FIG. 3 is a diagram of the system's basic set-up. Each sensor is installed into the exit line of the corresponding oil well. The signals obtained from each sensor are transferred to a control center, which is located in the oil extraction station. The control center deals with all the signals accumulated, including classified statistics, storage, and refurbishing. An alarm indicates to the observer that a delivery of feedback signals have been prepared and the corresponding command will be transmitted to the working site of the oil well at that point in time.

### DETAILED DESCRIPTION

Oil, water, and gas holdup are the major indexes for the quality control of crude oil extraction. Water holdup could reach colossal levels, in significant instances 100%, for instance in an extraction platform on the sea or a water flooding well. If the oils are continuously extracted in such a circumstance, the directional flow of the reservoir could be destroyed and will manufacture an oil well to the water well. To adjust the technology of the oil extraction, one requires the data of the status of crude oil underground. This status is obtained by continuously analyzing oil, water, and gas holdup of the extracted crude oil. The existing method for the analysis of crude oil is obtained by collecting oil samples by hand then analyzing the data in a laboratory. The analytical data of gas holdup is not sufficient due to the evaporation of hydrocarbons in the oil sample. The analysis of collecting samples by hand does not represent the complete status of the crude oil; therefore, the goal of optimum efficiency cannot be achieved. The present invention, an on-line monitoring method, will accurately obtain the oil, water, and gas holdup of the crude oil, and ensures that maximum efficiency for oil extraction will be fulfilled.

In general, a united station consists of several extraction stations, combining on average ten oil wells. Before being transferred to the united station, the crude oil from each well is joined together at the extraction station.

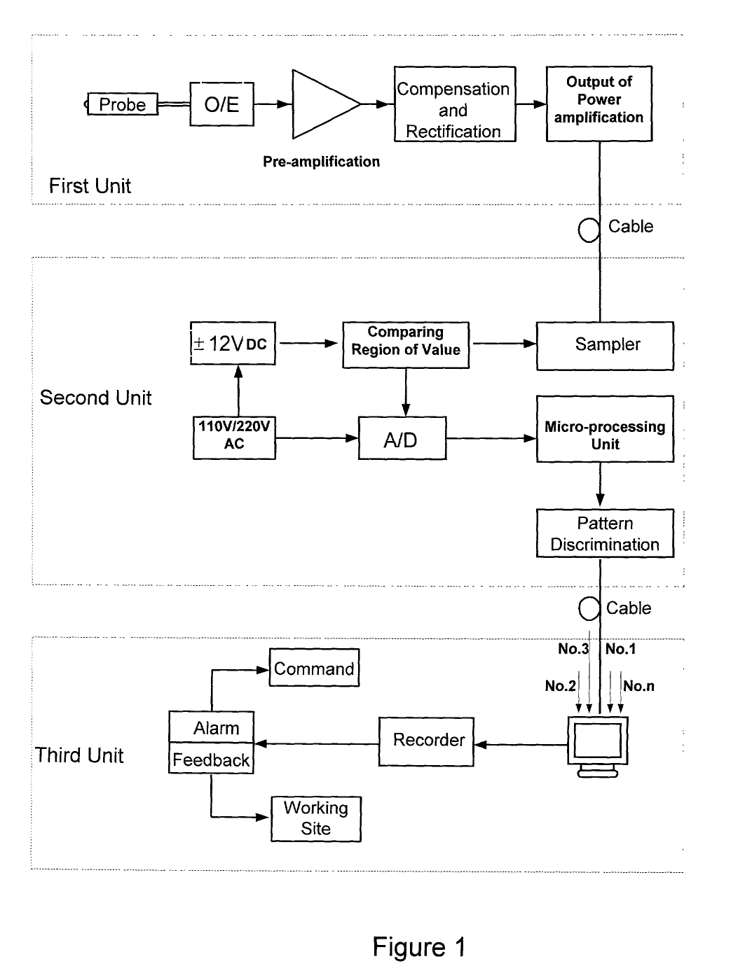

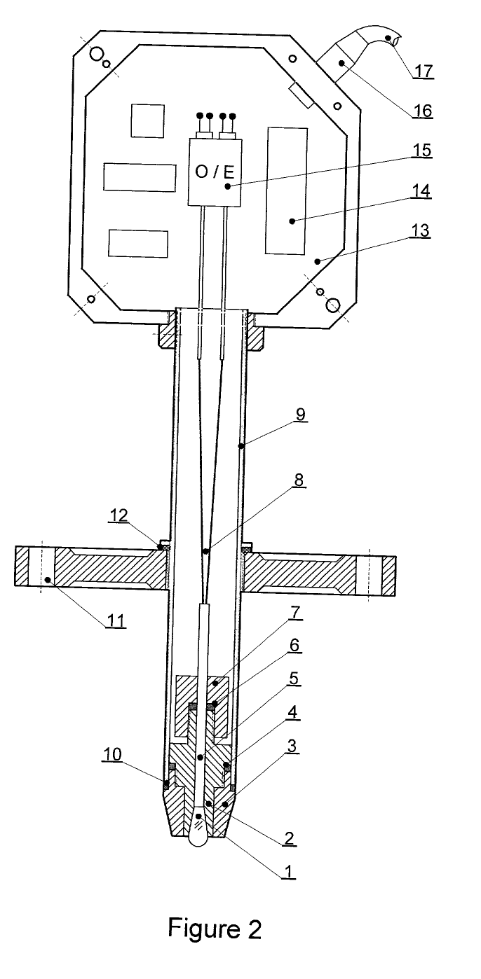

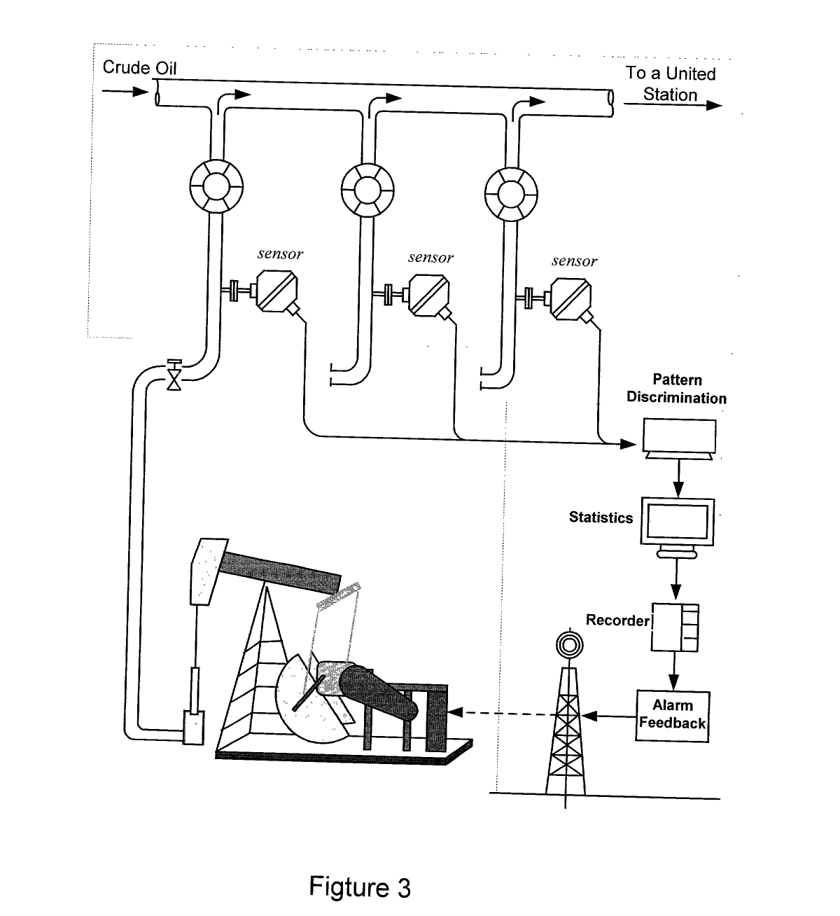

The present invention employs a method relating to the optics of the fiber sensors. Each fiber optical sensor is installed into the exit lines of the corresponding oil well. FIG. 1 is a schematic block diagram of the present invention, which consists of three units. The first unit is the sensor. The signals of oil/water/gas are obtained from the fiber optical sensor and are converted to electric signals. A cable to the second unit transmits the electric signals by pre-amplification, compensation, rectification, and through the output of power amplification. The second unit houses the 12 V DC power source to be used by the first unit. The sampler from the second unit transmits signals of oil/water/gas, which are obtained from the first unit from the micro-processing unit. Digital signals, after pattern discrimination, are transmitted to a computer through a cable. A computer, located in the third unit, contains all the data transmitted from the first and second units; which do classified statistics, recording, refurbishing, and feeding back signals. The computer provides data concerning oil, water, and gas holdup at any point in time such as a minute, an hour, a week, a month, or an extended period of time. The need will be based on what the customer demands. The third unit contains an alarm that will serve two separate functions. First the alarm will sound in a case where any abnormal activity is detected. Second, the alarm will deliver feedback signals and convey the corresponding command to the work site of the oil well to reach maximum operating conditions for oil extraction.

FIG. 2 shows the structure of the sensor of the present invention in further detail. Lights, with a wavelength range of 0.85-1.55, from the LED, are transmitted to the probe 1 through an optical fiber 8. The top end of the probe is immersed into an analyzed liquid. Any changes that occur in the refractive index of the space n in the analyzed liquid, accompanied by ratio changes of oil/water/gas in the analyzed liquid, will result in the adaptation of transmitted lights that passes by the probe 1. Based on the law of reflection, reflected light changes accordingly with the index n. The reflected lights are transformed to electric signals once received by a pin of the probe. The ratio signals of oil/water/gas are finally exported through amplification, compensation, and correction by a circuit plate 14. The multi-mold optical fiber 8 with its wick/cover ratio of 200/300 μm is constructed of quartz. A stainless steel pipe holds them together; the distance of two wicks is measures approximately 400 μm. Both ends of the fiber must be polished. The probe 1 is constructed of a blue gem or quartz with the taper of 1:50-1:10. The present invention pertains to a blue gem so that a greater load-resistance and a high refractive index can be achieved. The shape of the end of the gem can be either elliptical or spherical, or it can have a cone's surface. High resolution can be achieved by coating a layer of nano-materials (0.1-0.4 mm) that have a lower refractive index. The coating on the gem consists of 4-10 nm of nano-materials, such as gold or nickel to provide the sensor with a wide dynamic range. In further detail, the end of gem requires a pollution-resistant character, while maintaining high sensitivity. In the present invention we use electrophoresis to originate a coating on the metal surface. This coating consists of a polytetrafluroethylene (PFE) liquid combined with an addition of 1-5% epoxy resin; epoxy resin is classified as pollution-resistant and enhances the adhesive abilities of PFE liquid. The refractive index of the PFE layer ranges from 1.40 and 1.42.

The gem probe 1 is joins the stainless pipe 5 without an adhesive; the two parts are installed coaxially into the probe stand 2. The probe stand 2 is fixated onto the sensor stand 3, to prevent leakage a washer 4 is added. When the nut 7 is tightened up the lead washer 6 remains stationary. The optical fiber is secured by a secondary sealant. Similarly, a red copper washer is required when the stainless steel sleeve 9 is fastened to the sensor stand 3. Also, a sealing washer consisting of silicon rubber is required between the flange plate 11 and the flange inside of the pipe of the oil extraction. The circuit plate 14 is installed in the box 13, a housing for the circuit plate. Wires on the circuit plate 14 exit from the plug socket 16 and are connected to the cable 17. Each sensor corresponds to a particular pattern discrimination system.

Pattern discrimination is based on two schemes. The first scheme artificially determines the value of the refractive index of the analyzed liquid: 1-1.25 for gas, 1.26-1.39 for water, and 1.40-1.70 for oil. A distinction of three signals must be decided for oil, water, and gas. Each sample can claim one signal; the ratio obtained for occupancy/empty based on a sequence of pulse signals qualifies as the ratio of oil/water/gas at that particular point in time. The leading number is the frequency of the sample; the trailing number is the analytical error.

The second scheme's basis depends on the quality of oil, geographic location, and the condition of the oil well. The oil/water status of crude oil remains stable from the transfer underground to above ground; however, the micro bubbles and the dissolved gases in the oil may convert to considerably sized bubbles and dissociated gas. In any case, three statuses' can be obtained: oil predominated liquid, water predominated liquid, and gas predominated gas. Thus, the sample signals represent the following ratios: oil/water, water/oil, and gas/liquid. Each signal from the sample displays the percentage of the two components in a certain volume. The oil, water, and gas percentages in the cumulated volume, R_{O}, R_{W}, and R_{G}, can be obtained from the cumulated samples. In fact the cumulated volume also stands for the cumulated time, i.e. the ratios of R_{O}, R_{W}, and R_{G} are in units of time. Compared with the first scheme, clearly, the second scheme proves to be more accurate with a smaller margin for errors. Based on an individual customer's needs, the best scheme choice can be decided upon.

FIG. 3 is a graphical representation of the system, only an oil well drawing is shown. Take note that the present invention is not only suitable for use in the flowing well but also the oil platform on the sea.

What is claimed is:

1. A method and apparatus for the on-line monitoring of oil, gas, and water holdup. The apparatus comprises a series of fiber optical sensors that are installed into the transferring pipe of the crude oil. The signals of oil/water/gas ratios are obtained from the fiber optical sensor. The signals are transmitted to the micro-processing unit for pattern discrimination. A computer for statistics, storage, recording, inquiring, refurbishing, data feedback, and an alarm treats the signals of percentages of oil, water, and gas at the unit of time from the above system.

2. From claim 1, it can be acknowledged that the fiber optical sensors are installed into the exiting pipes of each oil well. It should be noted that a system can include several pattern discriminations, i.e. one combination box of pattern discriminations is used for a single oil well and has the capability to share with several wells. When the statistical system is shared, several sensors can be connected to form an intelligent managing network to maintain oil, water, and gas holdup. The network can support a series of oil wells or a single well.

3. In order to achieve pollution resistant goals, high levels of sensitivity, and high resolution, an Au or Ni nano-material layer with the thickness of 4-10 nm is coated on the end of the probe on the fiber optical sensor.
